(19) United States
(12) Patent Application Publication
(10) Pub. No.: US 2015/0374091 A1
(43) Pub. Date: Dec. 31, 2015

(54) DEPILATORY STRIP WITH EXFOLIATING BACKING

(71) Applicant: SI&D IP Pty Ltd., Baulkham Hills (AU)

(72) Inventor: Nadine Ismiel-Nash, Baulkham Hills (AU)

(21) Appl. No.: 14/752,580

(22) Filed: Jun. 26, 2015

(30) Foreign Application Priority Data
Jun. 27, 2014 (AU) 2014902475
Dec. 10, 2014 (AU) PCT/AU2014/050410

(51) Int. Cl. A45D 26/00; A61K 8/92; A61K 8/73; A61Q 9/04; A61K 8/85; A61K 8/81; A61K 8/02; A61K 8/31

(52) U.S. Cl. CPC A45D 26/0014; A61K 8/0208; A61K 8/922; A61K 8/927; A61K 8/31; A61Q 9/04; A61K 8/85; A61K 8/8111; A61K 8/731

(57) ABSTRACT

The present invention is concerned with hair removal from the human body and relates to improved depilatory wax strips and their application to hair removal by epilation. A depilatory strip is disclosed comprising an adhesive depilation paste, in particular a cold depilation wax formulation, deposited on one face of a backing sheet, in particular incorporating a non-woven fabric, characterised by the backing sheet having on its other face a coarse surface structure adapted to effect exfoliation of human skin and to remove dead skin cells to release ingrown hair prior to depilation.

### CROSS-REFERENCE TO RELATED APPLICATIONS

This Application claims priority to AU 2014902475 filed Jun. 27, 2014 and PCT/AU2014/050410 filed Dec. 10, 2014. Both applications are incorporated in their entirety.

### TECHNICAL FIELD

The present invention is concerned with hair removal from the human body and relates to improved depilatory wax strips and their application to hair removal by epilation.

### BACKGROUND OF THE INVENTION

The removal of body hair by epilation and depilation, particularly for cosmetic purposes, can be performed using several techniques.

One of the most commonly used epilation techniques, aimed at removing the entire hair strand, including parts below the skin surface in the follicles, involves the application of hot or cold depilatory wax formulations to an area of skin from which hair is to be removed, and the rapid pulling off of a strip of wax with hairs entrained in the set wax. In contrast to warm (or hot) wax epilation, where the wax is applied in liquid/pasty consistency directly onto the skin with a spatula or the like, and a textile strip is subsequently applied onto the wax and subsequently pulled-off to effect hair removal, cold wax formulations are pre-applied in a pasty state onto a backing strip of paper, textile (non-woven) or multi-film composition, and either covered with an easily removable protective film, or with a second backing strip so as to provide a two-in-one strip configuration. The wax formulation stays malleable and tacky during storage, and in most cases must be softened prior to use by gentle heating, eg by the user gently rubbing the strip and friction-heating it.

In use, the user removes the cover film protecting the wax layer from the strip, or separates the two backing films which sandwich the cold wax layer, presses the strip wax side down firmly onto the skin area to be epilated (and if need be rubs down the strip to increase penetration/embedding of hair stems in the wax layer), and then one end of the backing strip is grasped and pulled sharply away from the area to be epilated, thus removing hair entrained in the wax from the skin.

It will be appreciated that the interface between backing strip and wax is devised to provide sufficient adhesion between wax and the surface of the strip onto which the wax is applied, so that the wax is substantially retained on the backing material in the process of pulling off the backing strip from the skin surface. Considerable research has gone into this requirement, and an example of a prior art document directed to cold wax depilatory strips and which illustrates this is WO 96/21419 A1 of Reckitt & Coleman Products Ltd., the contents of which is incorporated herein by way of cross-reference. There are numerous different formats of cold wax strips on the Australian (and international) market, exemplified by Veet (brand) wax strips, Nad's (brand) wax strips, all of which are characterised by exhibiting backing strips devised to minimise wax remaining on the user's skin upon the strip being pulled from the user's skin.

In contrast to epilation, depilation is aimed at removing only the part of the hair above the skin surface. A typical depilation technique is shaving. Another technique used, though to lesser extent in western countries, involves the use of mitts and pads having an abrasive coating or sheet. In use, the pads/mitts are rubbed with vigour and a certain degree of pressure over the skin area in order to mechanically break down hair shafts without destroying or removing hair follicles. The hair stem above the skin is buffed away, during which process the skin surface is exfoliated too, the degree of exfoliation being in part a function of the coarseness of the abrasive/polishing surface.

In the context of this specification, unless the specific context requires otherwise, the terms epilation and depilation are used interchangeably, noting that body hair removal using application of wax onto skin and pulling the wax off the skin is commonly referred to as wax depilation.

One problem that arises with epilation is the formation of in-grown hair during hair growth after treatment. This poses a problem in that cold wax depilation treatments will not remove those hairs trapped under the skin. Another issue with cold wax depilation is that the user's skin ought to be cleaned (eg using soap or skin cleansing preparations) in order to remove excessive skin oils before application of the waxed backing strip, to achieve optimal epilation results. Further, in particular when hair removal is carried out at an early stage of hair re-growth, after a previous epilation operation, when hair stem length is short and hair growth is fine, cold wax hair removal tends to be less efficient. Without wanting to be tied down to the correct scientific explanation, this may be consequence of the fineness and smoothness of regrowing hair.

The present invention is aimed at providing in particular cold wax depilation strips with improved functionality.

### BRIEF SUMMARY OF THE INVENTION

In accordance with a first aspect of the invention, there is provided a depilatory strip comprising an adhesive depilation paste, in particular a cold depilation wax formulation, and first and second backing sheets, each preferably incorporating a non-woven fabric, wherein the backing sheets are removably joined to one another in facing relationship by a layer of the depilation paste sandwiched between them and which adheres to a first face of both backing sheets in such manner that peeling the backing sheets away from one another maintains a layer of said depilation paste on the first face of each of the backing sheets, characterised in that at least one of the backing sheets has on a second face opposite the first face a coarse surface structure with a roughness value sufficient and adequate to effect exfoliation of human skin.

During a typical manufacturing process of such depilatory strip, the wax is applied to the first face of one of the backing sheets only and then the second backing sheet is rolled over and sealed onto the first backing sheet. An alternative manufacturing process would see a thin layer of depilation paste applied onto the first face of both backing sheets which are subsequently brought into face abutting relationship and pressed together. In either case, on separating the strips, the depilation paste/wax will remain present on both sheets.

In accordance with a second aspect, the present invention provides a depilatory strip comprising an adhesive depilation paste, in particular a cold depilation wax formulation, deposited on a first face of a backing sheet which preferably comprises a non-woven textile (fabric), characterised by the backing sheet having on a second face opposite the first a coarse surface structure with a surface roughness value sufficient and adequate to effect exfoliation of human skin.

A protective film that is easily removable from the depilation paste, such as a silicon coated paper, would be applied onto the depilation paste to seal the strip off for packaging and storage purposes.

Adhesive depilation paste formulations, including cold depilation waxes, are known in the art and will not be described in detail herein.

Preferably, in the strip according to the first aspect, both backing sheets will have a coarse surface structure on the face opposite the one onto which the adhesive depilation paste is applied, whereby the coarse surface structure on the sheets may be the same or different as regards roughness values and/or topography. For the second aspect embodiment, while the backing sheet could have zones (surface areas) with different roughness values, it is preferred to provide a more or less uniform roughness across the entire coarse surface.

The term ‘coarse surface structure adapted to effect exfoliation of human skin’ is used in the present specification to denote the presence of a surface topography on the non-wax-carrying face of the backing sheet, provided by fine abrasive particles or strands on the exposed sheet face, that are conducive in effecting gentle skin exfoliation when the backing sheet is rubbed back and forth or in a circular motion (or in one direction) on human skin without undue pressure application. In addition and preferably, the coarse surface structure is also devised to have roughness values below which hair shafts are substantially degraded or entirely buffed away, as this would be detrimental to the subsequent cold wax epilation process which aims to remove the entire hair shaft including a subsurface portion, without breaking the hair shaft above the skin surface.

Advantageously, the coarse surface structure is devised such as to remove dead skin and release ingrown hair trapped under the skin, and cause a slight roughening or opening of the exterior surface (cuticle) of new-growth, fine hairs, with an aim of assisting in better embedding such finer hairs in the wax for subsequent epilation.

There are different ways in which the coarse surface structure may be implemented. In one embodiment, the structure is formed by a separate coating or film with abrasive particles applied onto a carrier sheet (substrate). In another, preferred embodiment, the coarse surface is formed by an exposed face of a heterogeneous non-woven textile substrate in which abrasive strands or particles are embedded in a pliable matrix substance whereby some of these strands or particles stand proud of a planar mean surface plane of the substrate faces.

Sanding paper may provide an illustration of one implementation of such coarse surface structure, although sanding paper is not a preferred embodiment; see below. Sandpaper contains numerous ‘sharp edges’ that are able to abrasively cut into a substrate surface (in the case of exfoliation this would be the very top human skin layer of the epidermis, the stratum corneum). The sharp edges are provided by particles glued onto or otherwise embedded into backing sheet material such as Kraft paper, textile backings or composite plastic film laminates, with a bonding agent in random or specific pattern(s). The grit size, or number of abrasive particles per surface area unit, in addition to the particle size and particle size distribution, are quality indicators affecting abrasion outcomes. The density of the grit is important too, noting that a more open-coated abrasive sheet surface has larger gaps between the particles, preventing abraded particles to gather and clog the surface and interfere with the abrasion process.

Accordingly, while a backing sheet embodiment of the waxing strip could exhibit the same type of abrasive media as used in conventional sanding paper, which use primarily aluminum oxide, garnet, silicon carbide, ceramic or similar inorganic/mineral abrasive particles, this is not a preferred embodiment. Softer abrasive media is preferred and selected to be less prone to roughen (or damage) the skin dermis when too much pressure is applied in the exfoliation/rubbing process.

Furthermore, given that the coarse surface structure is not intended to effect depilation per se but rather gentle skin exfoliation, the backing sheet is preferably manufactured to comprise a non-woven core layer onto/into which are adhesively deposited, coated or otherwise embedded particles, filaments and/or fibers of a material selected from cellulose, polymers (preferably bio-degradable), botanicals and combinations thereof The term ‘botanicals’ is used herein to denote plants and plant materials that are used in the manufacture of exfoliating particles and fibers, such as ground coffee, corn meal, jojoba beads, Aloe Vera fibers, ground coconut husks, etc.

It is preferred that the backing sheet comprises a non-woven textile with filaments/strands embedded in a polymer matrix to stand proud and create a ‘rough’ surface exhibiting surface roughness values conducive to skin exfoliation as defined above. In a preferred embodiment, such coarse surface structure will have roughness values in the range of Ra=15 to 38 μm, Rc=22 to 98 μm and Rz=77 to 210 μm. The surface uniformity (Rc/Rz) is preferably within the range of 40 to 50%.

The Ra value is the mean deviation of profile calculated by integrating the areas between the mean line (a line bisecting the roughness curve such that the areas above and below the line are equal) and the surface profile, and dividing by the evaluation length. Ra is the most commonly used one-dimensional roughness parameter, despite modern devices being able to provide a more detailed 2D and 3D characterisation of a ‘rough’ surface. Additional parameters give an informed user additional information that help characterise the roughness of a specimen surface. The Rc value is the average profile height, calculated by summing the height of each peak from the preceding valley, and dividing by the number of peaks along the evaluation length (one-dimensional value). The Rz value is the maximum height of profile calculated by summing the height of the maximum profile peak and the maximum valley depth in an evaluation length, with reference to the mean line.

In a preferred embodiment, the static coefficient of friction of the coarse surface structure is ideally in the range of 0.380 to 0.425, and the kinetic coefficient of friction of the coarse surface structure in the range of 0.305 to 0.340, tested by the method described in ASTM D1894-14 and using leather as the substrate material.

In one particularly preferred form, such non-woven textile will comprise a polymer matrix film, in particular PE, at basis weight of between 15 to 25, but preferably 20 to 22 g/m2, in which a mix of polyester, polypropylene and/or cellulose fibres at a basis weight of between 35 to 55, but preferably 45 g/m2 are embedded, for a total grammage of such non-woven textile sheet of between 50 to 80, but preferably 65 g/m2, with an average thickness of between 200 and 250, but preferably 230 μm.

A non-woven textile suitable to also perform its function as carrier for the wax formulation during epilation is one which has a tensile strength in lamination direction of between 1200 and 1600, but preferably 1400 N/m and in traverse direction of between 920 and 960, but preferably 940 N/m, with fracture elongation values at around 8% and around 16% in lamination and traverse direction, respectively, amongst other technical characteristics. The thickness of the backing sheet can be selected to have other values, as long as its flexibility in particular during removal of the strip from the skin in the epilation process, is ensured.

It will be understood by the skilled person that the abrasive filaments/fibers need to adhere to the backing sheet (substrate) or be embedded in its matrix in such manner that they will remain substantially attached to the sheet for the intended one-time use which cold wax strips are usually devised for.

It will be equally apparent to the skilled person that backing sheets for use in preferred embodiments of the invention could also be of multi-layer composite nature, such as described in the above mentioned patent publication WO 1996/021419 A1, always ensuring of course that a skin exfoliation layer provides an outer face of the waxing strip backing sheet.

Another preferred backing sheet embodiment will advantageously also be devised to comprise on the adhesive depilation paste carrying face a carrier film whose properties are optimized to provide the desired adhesiveness to ensure the wax remains attached to the backing sheet when it is peeled off the skin during the depilation process.

The present invention thus provides a single use product which allows the user to exfoliate the skin using the waxing strip itself, by gently rubbing it with its coarse surface structure against the skin, thus releasing any ingrown hair trapped beneath the skin and additionally removing skin surface impurities in the process. Furthermore, the rubbing process will generate heat and soften the wax formulation of the strip, so that once any protective film is removed from the adhesive depilation paste (eg the cold wax layer) or the two backing sheets of the strip are peeled from one another, the wax-carrying face of the backing sheet(s) can be applied onto the skin area for subsequent epilation. The exfoliation process will remove not only dead skin cells that trap hair and release ingrown hairs, but also other impurities and skin oils, and thus assist in preparing the skin for the cold wax application for the epilation process.

In another aspect of the present invention there is provided a method for beautification of human skin by first using a depilatory waxing strip in accordance with the first or second aspect of the invention to effect exfoliation of a desired area of a user's skin by rubbing, wherein the depilation paste is warmed by said rubbing action, then either removing the protective film of the strip of the first aspect or peeling-off the two backing sheets of the strip of the second aspect from one another, and then applying the backing sheet, depilation paste side down, unto the skin area, pressing the backing sheet down firmly and subsequently quickly stripping the backing sheet from the user's skin area to effect epilation.

Further preferred features of the invention will become apparent from the following illustrative description of a preferred embodiment of the present invention, provided with reference to the accompanying drawings.

### BRIEF DESCRIPTION OF THE DRAWINGS

FIG. 1 is an perspective view of a depilatory cold wax strip in a first embodiment of the invention, comprising two backing sheets, in which one sheet is shown partially peeled-off;

FIG. 2 is a schematic cross-section across the depilatory cold wax strip of FIG. 1;

FIG. 2A is an enlarged view of part of the depilatory cold wax strip showing the detail of the coarse surface structure;

FIG. 3 is an exploded view of a depilatory cold wax strip in a second embodiment of the invention, comprising two identical backing sheets; and

FIG. 4 is an exploded view of a depilatory cold wax strip in a third embodiment of the invention, comprising different types of backing sheets.

### DETAILED DESCRIPTION OF THE INVENTION

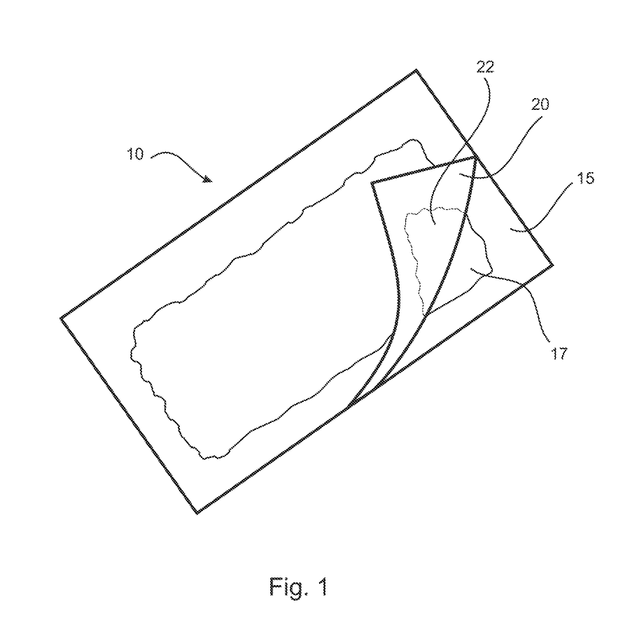

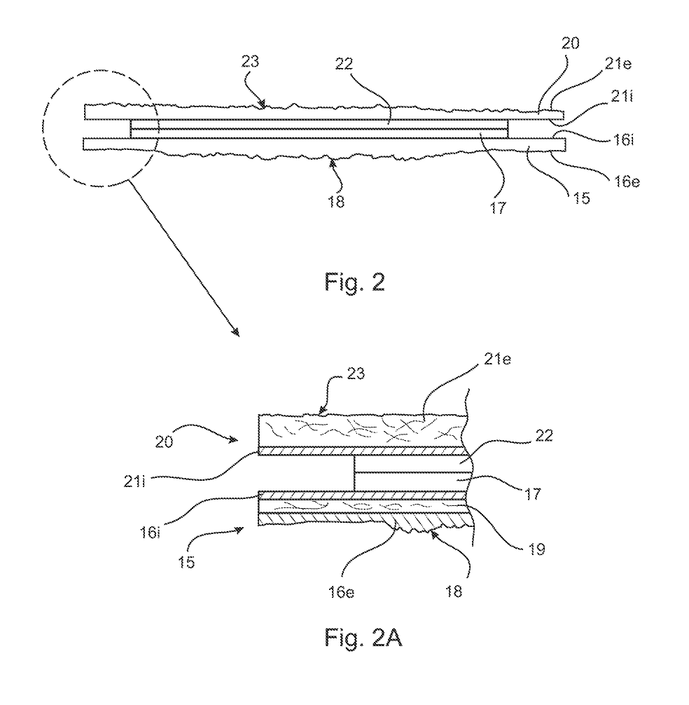

FIGS. 1 and 2 schematically illustrate a cold wax depilation strip 10 comprising two backing sheets 15, 20 onto each of which is applied on respective inner faces 16i and 21i a relatively thin layer 17, 22 of a cold wax depilation formula, and which are joined in wax facing relationship to one another by the tacky and pasty wax as is known in the art. This construction provides two useable depilation strips once the backing sheets 15, 20 are peeled-off each other.

The backing sheets 15, 20 are composite substrates, which may be of multilayer make-up, wherein the inner faces 16i and 21i are provided by a non-woven fabric or textile formulated to provide complementary adhesive properties to those of the cold wax depilation formula used for the strip 10 to ensure the wax layer 17, 22 remains attached to the respective backing sheets 15, 20 in the course of an epilation procedure, as is known. The non-woven or textile backing sheets may be coated with other substances, for example PE.

Relevantly, and in contrast to known cold wax depilatory strips, the exterior face or surface of the backing sheets 15, 20 is not smooth. In accordance with the invention, exterior face layers 16e and 21e of backing sheets 15, 20 comprise and define coarse surface structures 18, 23 suitable for skin exfoliation purposes.

It will be noted from the detailed enlargement of FIG. 2 that the layer make-up of the backing sheets 15 and 20 need not be the same for the upper sheet 20 and the lower sheet 15, although this would be preferred. Furthermore, it can be seen that the upper backing sheet 20 can be comprised of two distinct layers which provide the inner and exterior faces 21i and 21e of the non-woven textile backing sheet 20, respectively, although a single layer where the surface texture at the inner face 21i and at the outer (or exterior) face 21e are different can be used. The lower backing sheet 15 in FIG. 2 is shown to be comprised of three distinct layers, wherein a core component 19 may be a non-woven fabric onto which are permanently joined an inner non-woven, wax carrying layer or coating 16i on one face and an exterior non-woven textile layer 16e of a composition different than the core and inner sheet components.

In either case, the exterior faces (layers) 16e and 21e in the embodiment illustrated in FIG. 2 provide coarse surface structures 18 and 23 that are different to one another as regards coarseness. In other words, the topography of the exterior faces is different; so as to provide the strip 10 with two distinct exfoliation grits. The skilled reader will of course appreciate that the grits could be the same, if desired.

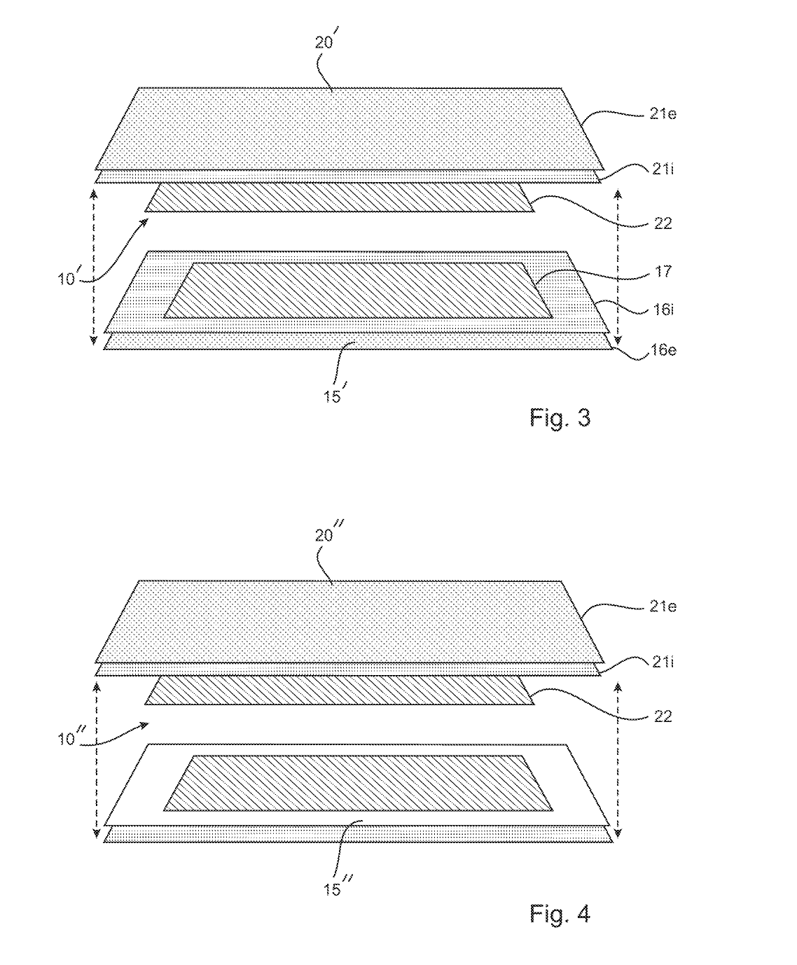

The embodiment of a waxing strip 10′ shown in exploded view in FIG. 3 comprises identical multi-layer backing sheets 15′ and 20′ comprised of an exfoliating external sheet 21e and 16e respectively bonded to an internal film 21i and 16i of polyethylene which carries wax layer 22 and 17. The exfoliating external sheet 21e and 16e has roughness values in the range of Rₐ=15 to 38 μm, R_{c}=22 to 98 μm, and R_{z}=77 to 210 μm, with a surface uniformity (R_{c}/R_{z}) of 40 to 50%. The static coefficient of friction of the coarse surface structure is in the range of 0.380 to 0.425, and the kinetic coefficient of friction of the coarse surface structure in the range of 0.305 to 0.340, tested by the method described in ASTM D1894-14 and using leather as the substrate material.

The embodiment of another waxing strip 10″ in accordance with the present invention shown in FIG. 4 is comprised of a standard or conventional waxing strip 15″ (comprised of an external, smooth PE film bonded to an interior non-woven depilatory backing sheet as per standard products available on the market) and another backing sheet 20″ of same construction as present in the embodiment of FIG. 2 and sheet 20′ of FIG. 3.

A depilatory strip 10 as described above with reference to FIGS. 1 and 3 has been manufactured by the applicant, using a depilatory cold wax composition formulated using viscoelastic materials based on rosin (colophony) and rosin derivatives, pliable film-forming agents based on bees wax and paraffin, and optional ingredients in small amounts selected from emollients, moisturisers, botanical extracts, skin soothing agents, skin smoothing agents, perfumes, colorants, preservatives, colour stabilisers, mica pigments and antioxidants.

The backing sheets 15 and 20 were selected to be identical in make-up and structure (and thus exhibiting the same coarseness at both exterior surface bodies 18 and 23).

These backing sheets are composite, non-woven laminates comprising a mix of polyester, polypropylene and cellulose fibres at a basis weight of 45 g/m2 in a PE matrix at basis weight of 20-22 g/m2 (for a total grammage of 65-67 g/m2), with an average thickness of 230 μm, a tensile strength in lamination direction of 1400 N/m and in traverse direction of 940 N/m, and fracture elongation values at 8% and 16% in lamination and traverse direction, respectively, amongst other technical characteristics.

Testing of depilatory strips in accordance with the different embodiments described above on a person in need of epilation has shown that by subjecting the skin zone to be epilated by gentle rubbing with the coarse surface of the backing sheet, buffing away dead skin and releasing ingrown hair as an essential function of the backing sheet (beyond that of carrying the wax formulation and allowing it to be removed with it in the epilation process), and also cleansing the skin of impurities (as a secondary function), thus creating a suitably prepared target zone for subsequent application of the wax present on the other face of the strip, with subsequent hair epilation resulting in a smooth hair-free skin area.

Moreover, the scope of the present application is not intended to be limited to the particular embodiments of the process, machine, manufacture, composition of matter, means, methods and steps described in the specification.

What is claimed is:

1. A depilatory strip, comprising:
an adhesive depilation paste comprising a cold depilation wax formulation;
a first backing sheet, comprising a non-woven substrate, having a first face on which the depilation paste is deposited;
wherein the first backing sheet has a second face opposite the first face which at least on a part thereof has a coarse surface structure adapted to effect exfoliation of human skin without depilation of hair from the exfoliated skin zone.

2. The depilatory strip according to claim 1, wherein the coarse surface structure of the second face has roughness values in the range of Ra=15 to 38 μm, Rc=22 to 98 μm and Rz=77 to 210 μm.

3. The depilatory strip according to claim 1, further comprising a second backing sheet, incorporating a non-woven substrate, having a first face, the first and second backing sheets removably joined to one another in facing relationship by sandwiching between them a layer of the depilation paste which adheres to both backing sheets in such manner that peeling the backing sheets away from one another maintains a layer of said depilation paste on the first face of each of the backing sheets.

4. The depilatory strip according to claim 3, wherein the second backing sheet has a second face opposite the first face which at least on part thereof has a coarse surface structure adapted to effect exfoliation of human skin without depilation of hair from the exfoliated skin zone.

5. The depilatory strip according to claim 4, wherein the coarse surface structure of the second face of the first and the second backing sheets have the same or different roughness values and/or topography.

6. The depilatory strip according to claim 5, wherein the coarse surface structure of the second face of the second backing sheet has roughness values in the range of Ra=15 to 38 μm, Rc=22 to 98 μm and Rz=77 to 210 μm.

7. The depilatory strip according to claim 1, wherein at least the first backing sheet comprises a non-woven composite with a matrix onto or into which are adhesively deposited, coated or embedded particles, filaments and/or fibers of a material selected from the group consisting of cellulose, polymers, bio-degradable polymers, botanicals and combinations thereof such as to provide the coarse surface.

8. The depilatory strip according to claim 1, wherein the coarse surface structure has a surface uniformity range (Rc/Rz) of 40 to 50%.

9. The depilatory strip according to claim 1, wherein the static coefficient of friction of the coarse surface structure is in the range of 0.380 to 0.425, and the kinetic coefficient of friction of the coarse surface structure in the range of 0.305 to 0.340, tested by the method described in ASTM D1894-14 and using leather as the substrate material.

10. A depilatory strip, comprising:
an adhesive depilation paste consisting of a cold depilation wax formulation; and
a backing sheet comprising a non-woven substrate, the backing sheet having a first face on which the depilation paste is deposited;
wherein by the backing sheet has a second face opposite the first face and which has at least on part thereof a coarse surface structure with surface roughness values selected to effect exfoliation of human skin, the coarse surface structure having roughness values in the range of Ra=15 to 38 μm, Rc=22 to 98 μm and Rz=77 to 210 μm.

11. The depilatory strip of claim 10, wherein the non-woven substrate is a composite comprising a polymer matrix onto or into which are adhesively deposited, coated or embedded particles, filaments and/or fibers of a material selected from the group consisting of cellulose, polymers, bio-degradable polymers, botanicals and combinations thereof to provide the coarse surface.

12. The depilatory strip of claim 1, wherein the first backing sheet(s) is a composite, non-woven laminate comprising a mix of polyester, polypropylene and cellulose fibres at a basis weight of 45 g/m2 in a PE matrix at basis weight of 20-22 g/m2, for a total grammage of 65-67 g/m2).

13. The depilatory strip of claim 12, wherein the backing sheet(s) has an average thickness of 230 μm, a tensile strength in lamination direction of 1400 N/m and in traverse direction of 940 N/m, and fracture elongation values at 8% and 16% in lamination and traverse direction, respectively.

14. The depilatory strip of claim 3, wherein the second backing sheet(s) is a composite, non-woven laminate comprising a mix of polyester, polypropylene and cellulose fibres at a basis weight of 45 g/m2 in a PE matrix at basis weight of 20-22 g/m2, for a total grammage of 65-67 g/m2).

15. The depilatory strip of claim 14, wherein the backing sheet(s) has an average thickness of 230 μm, a tensile strength in lamination direction of 1400 N/m and in traverse direction of 940 N/m, and fracture elongation values at 8% and 16% in lamination and traverse direction, respectively.

16. The depilatory strip of claim 11, wherein the backing sheet(s) is a composite, non-woven laminate comprising a mix of polyester, polypropylene and cellulose fibres at a basis weight of 45 g/m2 in a PE matrix at basis weight of 20-22 g/m2, for a total grammage of 65-67 g/m2).

17. The depilatory strip of claim 16, wherein the backing sheet(s) has an average thickness of 230 μm, a tensile strength in lamination direction of 1400 N/m and in traverse direction of 940 N/m, and fracture elongation values at 8% and 16% in lamination and traverse direction, respectively.

18. The depilatory strip of claim 3, wherein the first or second backing sheet comprises a PE-based polymer film bonded to the non-woven substrate to provide the wax-carrying first face of the backing sheet.
